(19) United States
(12) Patent Application Publication
(10) Pub. No.: US 2005/0063962 A1
(43) Pub. Date: Mar. 24, 2005

(54) Method of enucleation and oocyte activation in somatic cell nuclear transfer in primates

(75) Inventors: Soon Chye Ng, Singapore (SG); Naiqing Chen, Singapore (SG)

Correspondence Address: WILMER CUTLER PICKERING HALE AND DORR LLP, 60 STATE STREET, BOSTON, MA, 02109, US

(73) Assignee: National University of Singapore, Singapore (SG)

(21) Appl. No.: 10/889,225

(22) Filed: Jul. 12, 2004

Related U.S. Application Data
(60) Provisional application No. 60/486,366, filed on Jul. 11, 2003.

(51) Int. Cl. A61K 48/00; C12N 5/06; C12N 5/08

(52) U.S. Cl. 424/93.21; 435/366; 800/21; 435/364

(57) ABSTRACT

The present invention relates to a method for cell enucleation, comprising removal of the spindle body from a cell in metaphase with a minimal amount of cytoplasm from the cell, said method comprising using polarized light microscopy for visualization of the spindle body during cell enucleation. The present invention also relates to a method for production of an activated reconstructed vertebrate cell, said method comprising: a) culturing a reconstructed vertebrate cell for about 1.5 to about 3 hours after being reconstructed; b) applying at least one electrical pulse to the reconstructed cell; c) culturing the reconstructed cell for about a further 2 hours; and d) treating the reconstructed cell with a chemical activator. The methods of the invention find application in the preparation of totipotent or pluripotent cells of substantially identical genotype, as well as the cloning of vertebrates.

### FIELD OF THE INVENTION

The present invention relates to a new enucleation and oocyte activation method in somatic cell nuclear transfer in primates.

### BACKGROUND OF THE INVENTION

Somatic cell nuclear transfer (SCNT) is a powerful technique for preservation of endangered animals, multiplication of unique animal genotypes, and its application is being further expanded to the areas of transgenics, knock-in, or knock-out livestock. Basic understanding of cell dynamics in cancer can also be aided by SCNT research. Production of genetically identical animals would reduce the number of animals required for biomedical research and dramatically impact on studies pertaining to immune system function and early development of specific genetic diseases. Further application of SCNT research will also help clarify embryonic stem cell potentials. Although successful production of animal clones from somatic cells has been achieved in various species such as sheep, cattle, mice, goats, pigs, cats, and rabbits, there has been no success (not even reported pregnancies) in non-human primates.

To date, there is no efficient enucleation method in nuclear transfer. The standard technique is to use the dye Hoechst 33342 to stain the oocyte DNA, then Ultra-Violet (UV) exposure to determine the location of the stained DNA. The, spindle body is then aspirated according to epifluorescence imaging. As the oocyte is exposed to UV, the procedure may harm the oocyte. Another technique is to use a large enucleation needle to aspirate the 1^{st} polar body and the cytoplasm under the polar body, and the Karyoplast is then stained with Hoechst 33342, and checked under UV light for successful enucleation. The disadvantages include aspiration of a large volume of cytoplasm and uncertainty whether the spindle has been removed before checking under UV.

Activation method is another key factor affecting the success of SCNT. Many methods are used to activate oocytes, mostly direct current or chemical stimuli. The choice of activation appears to be dependent on the species. For example, Sr²⁺ is suitable for activation of reconstituted mouse embryos, but not for other species.

Quiescent G0 donor cells have been used during initial SCNT experiments. However, SCNT has also been achieved with donor cells in the G1 and G2/M phases. In mice, bovines and rabbits, there have been reports of spindle formation after somatic donor cell introduction into the enucleated oocyte, and of misaligned metaphase plates. More recently it has been suggested that the situation in primates is different to that in other animals, as disarrayed abnormal mitotic spindles with misaligned chromosomes were formed in all SCNT embryos, and no pregnancies resulted from SCNT embryos transferred into surrogates.

Thus, current investigated methods for somatic cell nuclear transfer in primates currently fail to provide successful activation of reconstructed embryos such that successful pregnancies can be achieved.

It is an object of the present invention to provide new methodology for use in somatic cell nuclear transfer procedures so as to enable successful activation of reconstituted non-human primate embryos.

### SUMMARY OF THE INVENTION

It is disclosed herein that (1) minimal ooplasm aspiration with removal of the spindle, and (2) the activation method and sequence are important in improving the efficiency and hence the success in somatic cell nuclear transfer in primates.

The present invention therefore provides new methods of enucleation and oocyte activation involving (1) An accurate non-harmful method of constant visualization of the spindle (using polarized light microscopy) allowing for minimal removal of the oocyte cytoplasm during enucleation and (2) A sequence of at least one electrical pulse with subsequent treatment with a chemical activator, such as ethanol or ionomycin for activation of reconstituted primate embryos.

Thus, according to a first embodiment of the invention, there is provided a method for cell enucleation, comprising removal of the spindle body with a minimal amount of cytoplasm from a cell in metaphase, said method comprising use of polarized light microscopy for visualization of the spindle body during cell enucleation.

According to another embodiment of the invention there is provided a method for production of an activated reconstructed vertebrate cell, said method comprising:
a. culturing a reconstructed vertebrate cell for a period of time after introduction of the donor nucleus to the recipient cell which is sufficient for the formation of the prematured condensed chromosome and spindle;
b. applying at least one electrical pulse to the reconstructed cell;
c. culturing the reconstructed cell for a period of time sufficient to allow the cell membrane to recover from the electrical pulse; and
d. treating the reconstructed embryo with at least one chemical activator.

The reconstructed cell may be produced by any appropriate means known in the art. For example, the reconstructed cell may be produced by introducing a donor vertebrate nucleus into an enucleated oocyte. The enucleated oocyte may be prepared by the cell enucleation method described above according to the first embodiment.

Totipotent or pluripotent cells isolated from activated reconstituted cells, wherein said cells are obtained by the above methods are also provided.

According to another embodiment of the invention, there is provided a method for generating a cloned vertebrate, said method comprising implanting an activated reconstructed vertebrate cell produced by a method of the invention into a compatible host uterus.

### DEFINITIONS

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

As used herein the term “adult cell” refers to any cell isolated from an adult animal, and may be isolated from any part of the animal, including, skin, kidney, liver, heart, follicle, and lung.

As used herein, the term “chemical activator” relates to compounds that are useful for non-electrical activation of reconstructed cells as known in the art. For example, suitable chemical activators may include: ethanol; inositol trisphosphate (IP₃); divalent ions (e.g., addition of Ca²⁺ and/or Sr²⁺); microtubule inhibitors (e.g., cytochalasin B); ionophores for divalent ions (e.g., the Ca²⁺ ionophore ionomycin); protein kinase inhibitors (e.g., 6-dimethylaminopurine (DMAP)); protein synthesis inhibitors (e.g. cycloheximide); phorbol esters such as phorbol 12-myristate 13-acetate (PMA); and thapsigargin.

As used herein the term “cloned” refers to a cell, embryonic cell, fetal cell, and/or animal cell having a nuclear DNA sequence that is substantially similar or identical to a nuclear DNA sequence of another cell, embryonic cell, fetal cell, and/or animal cell, and which has been generated by nuclear transfer means. The terms “substantially similar” and “identical” are described herein.

As used herein the term “comprising” means “including principally, but not necessarily solely”. Variations of the word “comprising”, such as “comprise” and “comprises”, have correspondingly varied meanings.

As used herein the term “cultured” or “culturing” in reference to cells refers to one or more cells that are may or may not be undergoing cell division in an in vitro environment. An in vitro environment may comprise any medium known in the art that is suitable for maintaining cells in vitro, such as suitable liquid medium or agar, for example. Specific examples of suitable in vitro environments for cell cultures are well known in the art, and are described in, for example, Culture of Animal Cells: a manual of basic techniques (3^{rd} edition, 1994, R. I. Freshney (ed.), Wiley-Liss, Inc.); Animal Cells: culture and media (1994, D. C. Darling, S. J. Morgan (eds), John Wiley and Sons, Ltd.); and Cells: a laboratory manual (vol. 1, 1998, D. L. Spector, R. D. Goldman, L. A. Leinwand (eds.), Cold Spring Harbor Laboratory Press); each of which is incorporated herein by reference in its entirety. Examples of cell culture media include, but are not limited to Dulbecco's Minimum Essential Medium (DMEM), and other readily available commercial media. Such media may contain one or more supplements such as serum (e.g., fetal calf serum) and/or one or more growth factors and/or cytokines as described herein.

As used herein the term “cumulus cell” refers to any cultured or non-cultured cell that is isolated from cells and/or tissue surrounding an oocyte. Suitable methods for isolating and culturing cumulus cells are well known in the art, and are discussed in, for example, Damiani et al. (1996), Mol. Reprod. Dev. 45: 521-534; Long et al. (1994), J. Reprod. Fert. 102: 361-369; and Wakayama et al. (1998), Nature 394: 369-373, each of which is incorporated herein by reference in its entirety.

The term “electrical pulse” as used herein refers to subjecting a nuclear donor and recipient oocyte to electric current. A nuclear donor and recipient oocyte can be aligned between electrodes and subjected to electrical current. Electrical current may be applied to cells as one pulse or as multiple pulses. Cells are typically cultured in a suitable medium for delivery of electrical pulses.

As used herein the term “embryo” or “embryonic” refers to a developing cell mass that has not implanted into an uterine membrane of a maternal host. Hence, the term “embryo” as used herein refers to a fertilized oocyte, a cybrid, a pre-biastocyst stage developing cell mass, a blastocyst, and/or any other developing cell mass that is at a stage of development prior to implantation into an uterine membrane of a maternal host. Embryos may not display a genital ridge. Hence, an “embryonic cell” is isolated from and/or has arisen from an embryo. An embryo can represent multiple stages of cell development, including: a zygote; a morula, or a blastocyst.

As used herein the term “fetal fibroblast cell” refers to any differentiated fetal cell having a fibroblast appearance, and may be identified and isolated by any suitable method as are well known in the art.

As used herein the term “fusion” refers to combination of portions of lipid membranes corresponding to a nuclear donor and a recipient oocyte. Lipid membranes can correspond to plasma membranes of cells or nuclear membranes, for example. Fusion can occur with addition of a fusion stimulus between a nuclear donor and recipient oocyte when they are placed adjacent to one another, or when a nuclear donor is placed in the perivitelline space of a recipient oocyte, for example. Fusion may be achieved by electrical or chemical means as are well known in the art.

As used herein the term “injection” in reference to embryos, refers to insertion into an oocyte, or the perivitelline membrane of an oocyte, of foreign material, including foreign nuclear material, typically with foreign cellular material associated with the foreign nuclear material, by any appropriate means, for example, insertion of a nuclear donor into the oocyte or perivitelline space. ‘Foreign material’ in this context, may include material obtained from a cell of the same species as the oocyte, but not from the same subject from which the oocyte is obtained.

As used herein the term “isolated” refers to a cell that is mechanically separated from another group of cells. Methods for isolating one or more cells from another group of cells are well known in the art, and are described in, for example, Culture of Animal Cells: a manual of basic techniques (3rd edition, 1994, R. I. Freshney (ed.), Wiley-Liss, Inc.); Animal Cells: culture and media (1994, D. C. Darling, S. J. Morgan, John Wiley and Sons, Ltd.); and Cells: a laboratory manual (vol. 1, 1998, D. L. Spector, R. D. Goldman, L. A. Leinwand (eds.), Cold Spring Harbor Laboratory Press).

As used herein the term “modified nuclear DNA” refers to a nuclear deoxyribonucleic acid sequence of a cell, embryo, fetus, or animal of the invention that has been manipulated by one or more recombinant DNA techniques, and may include nucleic acid material of non-primate origin.

As used herein the term “non-embryonic” or “somatic” cell refers to a cell that is not isolated from an embryo. Non-embryonic/somatic cells can be differentiated or non-differentiated, and can be nearly any somatic cell, such as cells isolated from an ex utero animal.

As used herein the term “nuclear transfer” refers to introducing a full complement of nuclear DNA from one cell to another cell, which may be enucleated before or after introducing the donor DNA. Typically, the recipient cell is enucleated before introduction of the donor DNA. Nuclear transfer methods are well known in the art, and are described in, for example, Nagashima et al. (1997), Mol. Reprod. Dev. 48: 339-343; Nagashima et al. (1992), J. Reprod. Dev. 38: 73-78; Prather et al. (1989), Biol. Reprod. 41: 414-419; Prather et al. (1990), Exp. Zool. 255: 355-358; Saito et al. (1992), Assis. Reprod. Tech. Andro. 259: 257-266; and Terlouw et al. (1992), Theriogenology 37: 309, each of which is incorporated herein by reference in its entirety.

As used herein the term “ploidy stabilizer” is any suitable compound as known in the art which inhibit or stabilize microtubule formation, directly or indirectly, and may include, for example: a microtubule inhibitor, such as cytochalasin B, nocodazole, colchicine or colcemid; a microtubule stabilizer, such as, for example, taxol; a protein kinase inhibitor such as 6-dimethylaminopurine (DMAP); a protein synthesis inhibitor such as cycloheximide; a phorbol ester such as phorbol 12-myristate 13-acetate (P MA); or thapsigargin.

As used herein the term “pluripotent” refers to a cell which, whilst not capable of dividing and differentiating in such a manner as to result in a live born animal or differentiating into any given cell type, is not fixed as to developmental potentialities, and has developmental plasticity, being capable of multiplying and differentiating into a plurality of cell types.

As used herein the term “reconstructed” refers to a cell which has been created by replacing the genetic material of a recipient cell with that from a donor cell. Other cellular components of the donor cell may also be transferred to the recipient cell, including the whole of the donor cell. A nuclear donor cell and a recipient oocyte can arise from the same species or different species. Any nuclear donor/recipient oocyte combinations are envisioned by the invention. The nuclear donor and recipient oocyte may be from the same genus, or from the same species. Cross-species nuclear transfer techniques can be utilized to produce cloned animals that are endangered or extinct.

As used herein the term “reprogramming” or “reprogrammed” refers to conversion of a cell into another cell having at least one differing characteristic, including another cell type that is not typically expressed during the life cycle of the former cell. For example, a non-totipotent cell can be reprogrammed into a totipotent cell, or a differentiated somatic cell can be reprogrammed into a totipotent cell.

As used herein the term “stem cell” refers to pluripotent or totipotent cells isolated from an embryo that are maintained in in vitro cell culture. Stem cells may be cultured with or without feeder cells, and may be established from embryonic cells isolated from embryos at any stage of development, including blastocyst stage embryos and pre-blastocyst stage embryos.

As used herein the term “substantially similar” in relation to nuclear DNA sequences relates to nuclear DNA sequences that are nearly identical. Differences between two sequences may arise as a result of copying errors or other modifications which may occur during replication of nuclear DNA. Substantially similar DNA sequences may be greater than 97% identical, more preferably greater than 98% identical, and most preferably greater than 99% identical. The term “identity” as used herein can also refer to amino acid sequences. It is preferred and expected that nuclear DNA sequences are identical for cloned animals.

As used herein the term “totipotent” refers to a cell capable of dividing and differentiating in such a manner as to result in a live born animal. The term “totipotent” may also refer to a cell that is capable of differentiating into any given cell type.

### DETAILED DESCRIPTION OF THE INVENTION

The present invention relates to new methods for use in cell enucleation and reconstructed primate embryo activation, and even more particularly in methods for somatic cell nuclear transfer and cloning of non-human primate animals. The methods of the invention involve (1) An accurate non-harmful method of constant visualization of the spindle (using polarized light microscopy) allowing for minimal removal of the oocyte cytoplasm during enucleation without exposing the cell(s) to UV light, and (2) A sequence of at least one electrical pulse with subsequent treatment with a chemical activator, such as ethanol or ionomycin for activation of reconstructed vertebrate cells. The studies described herein establish that (1) minimal ooplasm aspiration with removal of the spindle improves the likelihood of successful activation of reconstituted cells, and (2) the activation method and sequence are important for the efficiency and hence the success in somatic cell nuclear transfer in vertebrates.

1. Cell Enucleation

The present invention provides a method for cell enucleation, comprising removal of the spindle body with a minimal amount of cytoplasm from a cell in metaphase, said method comprising use of polarized light microscopy for visualization of the spindle body during cell enucleation.

Less than about 10%, less than about 9%, less than about 8%, less than about 7%, less than about 6%, less than about 5%, less than about 4%, less than about 3%, less than about 2%, less than about 1% or less than about 0.5% of the cytoplasm may be removed from said cell.

The cell may be an oocyte, including a vertebrate, mammalian, primate or non-human primate MII oocyte.

Where the cell is an oocyte, the spindle body may be removed through a small hole formed in the zona pellucida of the oocyte.

The hole needs to be minimal in size, yet still sufficient to allow removal of the spindle body from the cell, and may be from about 3 μm to about 30 μm, from about 4 μm to about 25 μm, from about 5 μm to about 20 μm, from about 5 μm to about 15 μm, from about 5 μm to about 10 μm or from about 5 μm to about 8 μm in diameter.

The hole may be made by any appropriate chemical or physical means permitting accurate perforation of the zona pellucida. Such methods may include zona drilling using acidic solutions, such as acid Tyrode's solution (NaCl, 0.8 g/100 ml; KCl, 0.02 g/100 ml; CaCl₂.2H₂O, 0.024 g/100 ml; MgCl₂.6H₂O, 0.01 g/100 ml; glucose, 0.1 g/100 ml; polyvinylpyrrolidone (PVP) 0.4 g/100 ml), or laser, or may include mechanical means, such as perforation using the needle for spindle body removal or other appropriate means. The small hole may be made by zona drilling using acid Tyrode's solution, the pH of which may be from about 2.5 to about 1.5, from about 2.3 to about 1.6, from about 2.1 to about 1.7, from about 1.9 to about 1.8, or about 1.8.

To facilitate accurate and controlled removal of the spindle body from the cell, the internal diameter of the needle is important. If it is too large, the volume of cytoplasm removed during enucleation, and concomitant cellular damage will be excessive. But if too small, the spindle may be difficult to aspirate: the spindle size is normally about 5×10 μm. The internal diameter of the needle may be the same size, or slightly greater in size than the spindle body and, if required, the hole made in the zona pellucida. The needle may have an internal diameter of from about 3 μm to about 30 μm, from about 4 μm to about 25 μm, from about 5 μm to about 20 μm, from about 5 μm to about 15 μm, from about 5 μm to about 10 μm or from about 5 μm to about 8 μm is used to aspirate the spindle body from the cell. The needle may be a non-spiked needle, and may have an internal diameter of from about 6 μm to about 10 μm is used to aspirate the spindle body from the cell.

2. Production of Activated Reconstructed Vertebrate Cells

The present invention also provides a method for producing an activated reconstructed vertebrate cell, said method comprising:
a. culturing a reconstructed vertebrate cell for a period of time after introduction of the donor nucleus to the recipient cell which is sufficient for the formation of the prematured condensed chromosome and spindle;
b. applying at least one electrical pulse to the reconstructed cell;
c. culturing the reconstructed cell for a period of time sufficient to allow the cell membrane to recover from the electrical pulse; and
d. treating the reconstructed embryo with at least one chemical activator.

The reconstructed cell may be produced by introducing a donor vertebrate nucleus into an enucleated cell.

The amount of time required after introduction of the donor nucleus to the recipient cell for the prematured condensed chromosome and spindle to form may vary from cell type to cell type and/or from species to species. In order to allow sufficient time for the prematured condensed chromosome and spindle to form, the cell may require culturing for from about 0.5 hours to about 10 hours, from about 1 hour to about 8 hours, from about 1.25 hours to about 6 hours, from about 1.5 hours to about 4 hours, from about 1.75 hours to about 3 hours, or about 2 hours after introduction of the donor nucleus to the recipient cell.

The at least one electrical pulse may be a direct current pulse, and may be applied at from about 50V/mm to about 300V/mm, from about 75V/mm to about 250V/mm, from about 100V/mm to about 225V/mm, from about 125V/mm to about 200V/mm, from about 130V/mm to about 180V/mm, from about 135V/mm to about 170V/mm, from about 140V/mm to about 160V/mm, from about 145V/mm to about 155V/mm, or about 150V/mm are applied to the reconstructed embryo. The duration of the at least one pulse of direct current may be applied for a period of from about 20 μs to about 100 μs, from about 25 μs to about 90 μs, from about 30 μs to about 80 μs, from about 35 μs to about 70 μs, from about 40 μs to about 60 μs, from about 45 μs to about 50 μs, or about 50 μs. Two pulses of direct current may be applied, and these may be from about 130 V/mm to about 180V/mm for from about 40 μs to about 60 μs each.

The amount of time for the cell membrane to recover from the at least one electrical pulse may vary from cell type to cell type and/or from species to species. However, as a guide, in order to allow the cell membrane to recover from the at least one electrical pulse, the cell may be cultured for from about 10 minutes to about 20 hours, from about 20 minutes to about 15 hours, from about minutes to about 10 hours, from about 40 minutes to about 8 hours, from about minutes to about 6 hours, from about 50 minutes to about 4 hours, from about hour to about 3 hours, or for about 2 hours after application of the electrical pulse, prior to treatment with a chemical activator.

Treatment with a chemical activator comprises culturing the reconstructed cell in culture medium comprising the chemical activator for sufficient time to activate the cell, followed by washing the cell with culture medium devoid of the chemical activator.

Where ethanol is used as the chemical activator, concentrations of ethanol for activation may be from about 1% v/v to about 50% v/v, from about 2% v/v to about 40% v/v, from about 3% v/v to about 30% v/v, from about 4% v/v to about 20% v/v, from about 5% v/v to about 10% v/v, from about 6% v/v to about 9% v/v, from about 6% v/v to about 8% v/v, or about 7% v/v.

Where ionomycin is used as the chemical activator, concentrations of ionomycin for activation may be from about 0.5 μM to about 50 μM, from about 1 μM to about 40 μM, from about 1.5 μM to about 30 μM, from about 2 μM to about 20 μM, from about 2.5 μM to about 10 μM, from about 3 μM to about 9 μM, from about 3.5 μM to about 8 μM, from about 4 μM to about 7 μM, from about 4.5 μM to about 6 μM, or about 5 μM.

The amount of time that cells are exposed to the chemical activator can also be modified to provide additional control over the activation process. The cells may be exposed to the chemical activator for between about 1 minute and about 30 minutes, between about 1.5 minutes and about 20 minutes, between about 2 minutes and about 15 minutes, between about 2.5 minutes and about 12 minutes, between about 3 minutes and about 10 minutes, between about 3.5 minutes and about 9 minutes, between about 4 minutes and about 8 minutes, between about 4 minutes and about 7 minutes, between about 4 minutes and about 6 minutes, or about 5 minutes.

Where ethanol or ionomycin is used as the chemical activator, the reconstructed cell may be treated with about 7% v/v ethanol or about 5 μM ionomycin for about 4 to about 7 minutes, typically 5 minutes.

In order to improve the likelihood of successful activation, the ploidy of the reconstructed cell should be maintained. Inhibition or stabilization of microtubule polymerisation is advantageous for preventing the production of multiple pronuclei, thereby maintaining correct ploidy. This can be achieved by culturing the cell with a ploidy stabilizer. Ploidy stabilizers are known in the art, and may include, for example: a microtubule inhibitor such as cytochalasin B, nocodazole, colchicine or colcemid; a microtubule stabilizer, such as, for example, taxol; a protein kinase inhibitor such as 6-dimethylaminopurine (DMAP); a protein synthesis inhibitor such as cycloheximide; a phorbol ester such as phorbol 12-myristate 13-acetate (PMA); or thapsigargin.

To improve the likelihood of successful activation, the cell should be cultured with the ploidy stabilizer at least after activation until pronucleus formation, and should be removed thereafter, typically before the first division takes place.

Thus, the method for producing an activated reconstructed vertebrate cell may also comprise culturing the activated reconstructed cell in the presence of at least one ploidy stabilizer.

The concentration of ploidy stabilizer required will depend on the stabilizer selected.

For example, where cytochalasin B is used as a ploidy stabilizer, the concentration of cytochalasin B used may be from about 1 μg/ml to about 50 μg/ml, from about 1.5 μg/ml to about 25 μg/ml, from about 2 μg/ml to about 20 μg/ml, from about 2 μg/ml to about 15 μg/ml, from about 2.5 μg/ml to about 10 μg/ml, from about 3 μg/ml to about 9 μg/ml, from about 3.5 μg/ml to about 8 μg/ml, from about 4 μg/ml to about 7 μg/ml, from about 4.5 μg/ml to about 6 μg/ml, or about 5 μg/ml.

Where cycloheximide is used as a ploidy stabilizer, the concentration of cycloheximide used may be from about 1 μg/ml to about 100 μg/ml, from about 2 μg/ml to about 50 μg/ml, from about 3 μg/ml to about 40 μg/ml, from about 4 μg/ml to about 30 μg/ml, from about 5 μg/ml to about 20 μg/ml, from about 6 μg/ml to about 18 μg/ml, from about 7 μg/ml to about 16 μg/ml, from about 8 μg/ml to about 14 μg/ml, from about 9 μg/ml to about 12 μg/ml, or about 10 μg/ml.

The reconstructed cell may be cultured with more than one ploidy stabilizer, for example with both cytochalasin B and cycloheximide.

The reconstructed cell may be cultured with the ploidy stabilizer for from about 1 hour to about 20 hours, from about 2 hours to about 18 hours, from about 2.5 hours to about 16 hours, from about 3 hours to about 14 hours, from about 3.5 hours to about 12 hours, from about 4 hours to about 10 hours, from about 4.5 hours to about 8 hours, or from about 5 hours to about 6 hours.

2.2 Recipient Cells

Recipient cells for use in the SCNT procedures of the present invention may be any suitable cell, including oocytes, and MII oocytes.

Oocytes can be isolated from oviducts and/or ovaries of live or deceased animals by oviductal recovery procedures or transvaginal oocyte recovery procedures well known in the art and described herein.

If necessary, oocytes can be matured in a variety of media well known to a person of ordinary skill in the art, and may be cryopreserved and then thawed before placing the oocytes in maturation medium. Components of an oocyte maturation medium can include molecules that arrest oocyte maturation. Cryopreservation procedures for cells and embryos are well known in the art as discussed herein.

The recipient cell may be from any vertebrate, including mammals selected from the group consisting of human, non-human primate, mice, cattle, sheep, goats, horses, rabbits, birds, cats and dogs. More typically, the vertebrate is human, or non-human primate. Even more typically, the vertebrate is non-human primate.

2.3 Donor Nucleus

The donor nucleus may be any suitable somatic cell nucleus, including a cumulus cell nucleus or fibroblast cell nucleus, and may be from a quiescent somatic cell in the G0 or G1 phase.

The donor cell may be from any vertebrate, including mammals selected from the group consisting of human, non-human primate, mice, cattle, sheep, goats, horses, rabbits, birds, cats and dogs. More typically, the vertebrate is human, or non-human primate. Even more typically, the vertebrate is non-human primate.

Both the oocyte and the donor nucleus may be of primate origin.

A nuclear donor cell and a recipient oocyte can arise from the same species or different species. Any nuclear donor/recipient oocyte combinations are envisioned by the invention. The nuclear donor and recipient oocyte may be from the same genus, or from the same species. Cross-species nuclear transfer techniques can be utilized to produce cloned animals that are endangered or extinct.

Cells for use in the methods of the present invention may be synchronised within a same stage of the cell cycle, and about 50%, about 70%, or even about 90% of cells in a population of cells may be arrested in one stage of the cell cycle. Cell cycle stage can be distinguished by relative cell size as well as by a variety of cell markers and methods well known in the art. Cells may be synchronized by arresting them (i.e., cells are not dividing) in a discrete stage of the cell cycle.

A nuclear donor may be injected into the cytoplasm of an oocyte. This direct injection approach is well known to a person of ordinary skill in the art. For a direct injection approach to nuclear transfer, a whole cell may be injected into an oocyte, or alternatively, a nucleus isolated from a cell may be injected into an oocyte. Such an isolated nucleus may be surrounded by nuclear membrane only, or the isolated nucleus may be surrounded by nuclear membrane and plasma membrane in any proportion. An oocyte may be pre-treated to enhance the strength of its plasma membrane, such as by incubating the oocyte in sucrose prior to injection of a nuclear donor.

The donor nucleus may be introduced into the enucleated oocyte by direct injection, and may be introduced into the enucleated oocyte by direct injection of the whole donor cell into the oocyte.

Alternatively, the donor nucleus may be introduced into the enucleated oocyte by electrofusion of the enucleated oocyte with the donor cell.

A nuclear donor can also be placed into the perivitelline space of an oocyte for translocation into the oocyte. Techniques for placing a nuclear donor into the perivitelline space of an enucleated oocyte are well known in the art.

At least a portion of plasma membrane from a nuclear donor and recipient oocyte can be fused together by utilizing techniques well known in the art, and described in, for example, Willadsen (1986), Nature 320:63-65, hereby incorporated herein by reference in its entirety. Typically, lipid membranes can be fused together by electrical or chemical means.

Processes for fusion that are not explicitly discussed herein can be determined without undue experimentation. For example, modifications to cell fusion techniques can be monitored for their efficiency by viewing the degree of cell fusion under a microscope. The resulting embryo can then be cloned and identified as a totipotent embryo by methods well known in the art, which can include tests for selectable markers and/or tests for developing an animal.

A typical method for producing an activated reconstituted vertebrate cell by the methods of the present invention is as follows.

A polarized light microscopy system is used to check for the presence and the position of the spindle of an MII oocyte. Acid Tyrode's solution (pH=1.8) is used to dissolve the zona pellucida to create a small opening (needle ID: 2-3 μm; hole ID: 5-8 μm); finally, a non-spiked needle (ID: 8-10 μm) is used to aspirate the spindle with minimal ooplasm. The total volume aspirated is about 0.1-0.5% of the whole oocyte.

[image]

The somatic cell is subsequently introduced into the enucleated oocyte by direct injection or by electro-fusion.

(2) Activation:

a. Direct current is applied to pulse the reconstructed eggs
b. Approximately 2 h later, exposure to chemical activator (e.g., ethanol or ionomycin) for about 5 min
c. Culture the reconstituted embryo for about 5 h in medium with Cycloheximide and cytochalasin B (CXCB)

Two hours after injection of the donor cell, the reconstructed oocyte is activated with direct current (50 μs, 150V/mm, 2 pulses), after about another 2 h culture, the reconstituted embryo is treated with 7% v/v ethanol for about 5 min, then cultured in culture medium supplemented with 10 μg/ml of cycloheximide and 5 μg/ml of cytochalasin B for 5 h.

According to the invention, the donor nucleus for use in producing an activated reconstituted primate cell may comprise modified nuclear DNA. Modified nuclear DNA includes a DNA sequence that encodes a recombinant product which may provide a beneficial or advantageous phenotypic result in the resultant cell(s), and or which is of value when expressed by, and/or isolated from cultured cell(s), and is typically a polypeptide or a ribozyme. The recombinant product may be expressed in a biological fluid or tissue. The modified nuclear DNA comprises at least one other DNA sequence that can function as a regulatory element, typically selected from the group consisting of promoter, enhancer, insulator, and repressor.

Recombinant DNA techniques are well known in the art, and may include inserting a DNA sequence from another organism into target nuclear DNA, deleting one or more DNA sequences from target nuclear DNA, and introducing one or more base mutations (e.g., site-directed mutations) into target nuclear DNA. Methods and tools for insertion, deletion, and mutation of nuclear DNA of mammalian cells are well-known to a person of ordinary skill in the art, and are described in, for example, Molecular Cloning, a Laboratory Manual (2^{nd} Ed., 1989, Sambrook, Fritsch, and Maniatis, Cold Spring Harbor Laboratory Press); U.S. Pat. No. 5,612,205, “Homologous Recombination in Mammalian Cells,” Kay et al., issued Mar. 18, 1997; PCT publication WO 93/22432, “Method for Identifying Transgenic Pre-Implantation Embryos”; and WO 98/16630, Piedrahita & Bazer, published Apr. 23, 1998, “Methods for the Generation of Primordial Germ Cells and Transgenic Animal Species,” each of which is incorporated herein by reference in its entirety.

Advantageously, use of such recombinant techniques can result in transgenic cells, embryos, fetuses, or animals in which one or more genes have been “knocked out”, in which said gene(s) is/are no longer expressed in a functional manner.

3. Pluripotent/Totipotent Cells

The present invention also provides totipotent or pluripotent cells isolated from activated reconstituted vertebrate cells, wherein said cells are obtained by the SCNT methods of the invention.

A cell resulting from a nuclear transfer process may be manipulated in a variety of manners. The invention relates to cloned cells that arise from at least one nuclear transfer.

If the cells are embryonic cells arising from an activated reconstructed cell produced by the methods of the invention, these may be disaggregated and utilized to establish cultured cells, referred to as embryonic stem cells or embryonic stem-like cells. The embryonic stem cells can be derived from early embryos, morulae, and blastocyst stage embryos. Methods for producing cultured embryonic cells are well known in the art.

If embryos are allowed to develop into a fetus in utero, cells isolated from that developing fetus, including primordial germ cells, genital ridge cells, and fetal fibroblast cells can be utilized to establish cultured cells.

Cloned pluripotent or totipotent cells resulting from nuclear transfer can also be manipulated by cryopreserving and/or thawing the embryos. Other manipulation methods include in vitro culture processes; performing embryo transfer into a maternal recipient; disaggregating blastomeres for nuclear transfer processes; disaggregating blastomeres or inner cell mass cells for establishing cell lines for use in nuclear transfer procedures; embryo splitting procedures; embryo aggregating procedures; embryo sexing procedures; and embryo biopsying procedures. The exemplary manipulation procedures are not meant to be limiting and the invention relates to any embryo manipulation procedure known in the art.

4. Cloned Vertebrates and Methods Therefor

According to another embodiment of the invention, there is provided a method for generating a cloned vertebrate, said method comprising implanting an activated reconstructed vertebrate cell produced by a method of the invention into a compatible host uterus.

The invention will now be described in greater detail by reference to specific Examples, which should not be construed as in any way limiting the scope of the invention.

### EXAMPLES

Materials and Methods

Animals

Mature female Long-tailed Macaques (M. fascicularis) weighing between 2.0 and 2.5 kg were made available for this study by the Wildlife Reserves Singapore. Monkeys were kept in a holding enclosure prior to being housed in individual cages in a well ventilated room with an average room temperature of 28° C. and 12 h daylight for daily treatments of gonadotropins. Monkeys were fed a mixed diet of fresh fruits and vegetables, supplemented with commercially available monkey chow and water. All animal procedures were approved by the Animal Holding Unit, Faculty of Medicine, National University of Singapore and the Department of Veterinary, Conservation and Research, Wildlife Reserves Singapore.

Establishment and Culture of Donor Cell Tissue Sources

Skin biopsy specimens were derived from a 180-day-old male Long-tailed Macaque (M. fascicularis) fetus and an adult male Lion-tailed Macaque (M. silenus). Cumulus cells were obtained from the follicles of the macaques from which oocytes were removed. Fresh cumulus cells were used as donor cells without further treatments.

Establishment and Culture of Fibroblast Cells

Skin biopsy specimens were washed in Ca²⁺-/Mg²⁺-free Dulbecco PBS (PBS; Invitrogen) and minced into pieces. Tissue pieces were planted onto the bottom of 4-well dishes (Nunc, Denmark) before adding Dulbecco modified Eagle medium (DMEM; Invitrogen) supplemented with 100 IU/ml of penicillin, 100 mg/ml of streptomycin (Sigma), and 10% (v/v) fetal bovine serum (FBS; Invitrogen) and cultured at 37° C. in 5% CO₂. Tissue pieces were removed using 30G needle (BD) when cells with fibroblast-like morphology started to migrate out of the tissues. After reaching 100% confluency, monolayers of cells were disaggregated using PBS containing 0.15% (w/v) trypsin and 1.8 mM EDTA and passaged two more times before being frozen in DMEM with 20% FBS and 10% (w/v) dimethyl sulfoxide (Sigma) and stored in liquid nitrogen.

Fibroblasts Treatments and Flow Cytometric Analysis of the Cell Cycle

The cell cycle comparisons of primary fibroblasts were made between cycling, serum-starved cells and cells that were cultured to confluency. Cell culture flasks (75 cm³ volume) were plated with frozen/thawed fibroblasts at a concentration of 1-3×10⁶ cells/flask. After reaching 70%-80% confluency, cycling cells were fixed in ethanol as described below. These cells were used as controls for comparison purposes.

Other cells were grown to 100% confluency and then allocated to one of the following treatments before being fixed: (a) replacement of growth medium with DMEM+0.5% FBS and culture for an additional 2 or 5 days (serum starvation); or (b) changing of regular growth medium every 2-3 days for an additional 2 or 5 days of culture (contact inhibition).

For fixation, cells from each treatment were disaggregated as described above, pelleted by centrifugation (5 min at 130×g), resuspended in 0.5 ml of PBS, and slowly mixed with 4.5 ml of cold, 70% (v/v) ethanol. After at least 12 h of ethanol fixation at 4° C., cells were pelleted, washed twice with PBS, and stained in PBS containing 0.1% (v/v) Triton X-100, 0.2 mg/ml of RNase A, and 20 mg/ml of propidium iodide (Sigma, USA) for 15 min at 37° C. Stained cells were then filtered through a 30-mm nylon mesh (Sefar, Switzerland) and analyzed with an Epics-Elite flow-analyzer (Coulter, USA). Percentages of cells existing within the G₀/G₁, S, and G₂/M phases of the cell cycle were calculated using Winmdi version 2.8 based on the PMT4 histogram.

Ovarian Stimulation and Macaque Oocyte Recovery

Procedures for superovulation of the Long-tailed Macaque (LoTM, M. fascicularis) and collection of their oocytes have been described previously (Ng, S. C., Martelli, P., Liow, S. L., Herbert, S. & Oh, S. H., Theriogenology 58, 1385-1397 (2002)). Briefly, cycling female monkeys were hyperstimulated with a GnRH agonist, triptorelin (Decapeptyl, Ferring, Kiel, Germany) for two weeks, then human recombinant follicle stimulating hormone (rFSH; Gonal-F, 75IU, Serono, Geneva) was administered for 12 days. On the last day of the FSH treatment, human chorionic gonadotropin (hCG; Profasi, Serono, Geneva) was administered. Cumulus-oocyte complexes were collected from anesthetized animals by laparoscopic follicular aspiration (34-36 h after hCG administration) and placed in TALP (modified Tyrode solution with albumin, lactate, and pyruvate) medium (Bavister, B. D. & Yanagimachi, R., Biol. Reprod. 16, 228-237 (1977)) containing 0.3% BSA (TH3) in an incubator at 37° C., 5% CO₂ in air. Oocytes stripped of cumulus cells by mechanical pipetting after brief exposure to 80IU/ml of hyaluronidase (Sigma, USA) were placed in medium IVF-20 (Vitrolife, Sweden) where they were kept in an incubator at 37° C. in 5% CO₂ until further use.

SCNT Procedures

Enucleation. Recipient MII oocytes were loaded individually into small droplets, 5 μl of HEPES buffered IVF medium (Ferticult, Belgium) containing 10 μg/ml cytochalasin B (Sigma, Mo., USA), on the glass bottom dish (Bioptechs, Butler, Pa.). A small hole in the zona pellucida (ZP) was made with acidic Tyrode solution (pH=1.8) using a 5 μm I.D. homemade pipette, followed by enucleation using an 8 μm I.D. non-spiked homemade pipette to aspirate the second meiotic spindle under polarized light microscopy (SpindleView, Cambridge Research Instrument Inc., MA).

Nuclear Transfer. LoTM fresh cumulus and starved fetal skin fibroblast cells, as well as starved Lion-tailed Macaque (LiTM) adult skin fibroblast cells were used as donor cells for nuclear transfer. Single donor cells were picked up from ICSI-100 (Vitrolife, Sweden), then directly microiniected into enucleated oocytes through the opening in the previously “drilled” zona pellucida. 8 μm and 5 μm I.D. spiked pipettes were respectively used for fibroblast and cumulus cells.

Activation was induced between 2-4 h after cell microinjection by electric pulses followed about two hours later by treatment with culture medium comprising 5 μM ionomycin (Sigma) or 7% ethanol for 5 min. Two consecutive direct current pulses (1.5 kV/cm, 50 μsec) were delivered by a BTX Cell Manipulator 2001 (Genentronics, Inc., San Diego, Calif.) in HEPES-buffered IVF medium containing 10% FCS (Sigma).

SCNT Embryo Culture

All SCNT embryos were cultured in medium IVF-20 (Vitrolife, Sweden) after manipulation and maintained in a moisture incubator at 37° C. with 5% CO₂, 5% O₂ and 90% N₂. After activation, SCNT embryos were cultured in IVF-20 containing 5 μM cytochalasin B and 10 μg/ml cycloheximide for 5 h, then transferred to medium IVF-20 after washing 4 times. 14-16 h after activation, nucleus formation was checked before transfer to pre-equilibrated medium G1.2 (Vitrolife, Sweden). 24 h later, all SCNT embryos were checked and transferred to medium G2.2 (Vitrolife, Sweden). After culture for another 28-30 h, selected 4-8 cell SCNT embryos were placed into recipient oviducts laparoscopically.

Embryo Transfer and Pregnancy Monitoring

Procedures for embryo transfer of reconstructed embryos has been described previously (Ng, S. C., Martelli, P., Liow, S. L., Herbert, S. & Oh, S. H., Theriogenology 58,1385-1397 (2002)). Briefly, 2 to 5 SCNT embryos were laparoscopically placed into the fallopian tube of a monkey from which oocytes were recovered earlier. The embryos were transferred to medium G2.2 and aspirated into a pre-rinsed, self-made embryo transfer catheter controlled by a 1-cc syringe through a 25 g hypodermic needle. The tip of the catheter was inserted 1-cm deep into the oviduct, and embryos expelled. Luteal phase support was provided by 10 mg progesterone administered intramuscularly for 14 days starting on the day of OR. Pregnancies were ascertained by fetal ultrasound with the presence of a viable gestational sac and heart beat⁴⁶.

Imaging

At different time points following injection and activation, reconstructed oocytes were processed for immunocytochemical staining to observe cytoskeletal organization and DNA configuration. Microtubules and DNA were detected as described previously²⁰. Briefly, the oocytes were permeabilized in modified buffer M for 20 min, fixed in methanol at −20° C. for 10 min and stored in solution at 4° C. for 1-7 days. Fixed oocytes were incubated for 90 min at 38.5° C. with 1:300 dilution of anti-α-tubulin antibody (Sigma, USA) in PBS. After several washes, oocytes were incubated in a blocking solution for 1 h at 38.5° C., followed by incubation with 1:200 dilution of FITC-labeled goat anti-mouse antibody (Sigma, USA) in PBS. DNA was fluorescently detected by exposure to 50 μg/ml of propidium iodide DNA stain for 30 min. Controls included non-immune and secondary antibodies alone, which did not detect spindle. Slides were examined using laser-scanning confocal microscopy. Microtubules were detected using α-tubulin antibody. Laser-scanning confocal microscopy was performed using a Zeiss LSM500 equipped with Argon and Helium-Neon lasers for the simultaneous excitation of FITC-conjugated secondary antibodies (Sigma) and propidium iodide DNA stain.

Statistical Analysis

Results were analyzed using the Pearson's Chi-squared test. A P value of <0.05 was considered to be statistically significant.

Results

Nuclear Formation and First Cell Division of SCNT with Three Types of Donor Cells

A total of 1108 oocytes were retrieved from 32 Cynomolgus monkeys, or Long-tailed Macaques (LoTM, Macaca fascicularis) in 71 cycles by laparoscopy. 62.8% (696/1108) of these oocytes were matured (MII) oocytes, 95.9% oocytes (497/518; the remaining 178 MII oocytes were used for other experiments) were successfully enucleated for SCNT under polarized microscopy. 94.8% of the enucleated oocytes were successfully microinjected with three types of somatic cells: LoTM cumulus; LoTM fetal skin fibroblasts; and Lion-tailed macaque (LiTM, Macaca silenus) adult skin fibroblasts. After 2 days, 5 days and 8 days of serum starvation, 62%, 66%, 77% and 74% of the fibroblasts were in G1/G0, respectively. Table 1 shows the results of nuclear formation and first cleavage after activation using three types of donor cells. Nuclear formation and normal division of SCNT embryos were not markedly affected by donor cell types, being similar among the three different types of donor cell: cumulus, fetal fibroblast and cross-species adult fibroblast. Interestingly, two-nuclei formation rate was significantly higher in iso-species nuclear transfer (cumulus & fetal skin fibroblast) then hetero-species nuclear transfer (adult skin fibroblast), and abnormal divisions were significantly lower in iso-species NT than in hetero-species nuclear transfer.

Spindle Formation in NT Embryos (Table 2)

Microtubule assembly and DNA changes of reconstructed oocytes were examined by fixing at the different time-points after the somatic cells were introduced into the enucleated oocytes. There was minimal change in somatic DNA and no microtubule assembly within the first 30 min after cell injection. Within two hours after activation, the prematurely condensed chromosomes segregated and moved towards the two spindle poles thus forming two nuclei. Fifty-four reconstructed oocytes were fixed at 2 h after cell injection. 70.4% (38/54) of somatic cell DNA underwent condensation to form premature condensed

TABLE 1
Efficiency of SCNT in Macaques

| | | Cell Injection | | | | | | | | | | | |
|---|---|---|---|---|---|---|---|---|---|---|---|---|---|
| | | No. of | No. of | Successful | No. of | Nuclear Formation % | | | | | | | |
| Donor Cell | | Enucleated | Injected | transfers | Injected | With | | | | 1^{st} Cell Division % | | | |
| Species† | Cell type | Oocytes | Oocytes | % | Oocytes* | RN‡ | 1 RN | 2 RN | ≧3 RN | Norm. | Abnor | 1 cell | Frag. |
| LoTM | Cumulus | 195 | 190 | 97.4 | 155 | 33.5 | 11.0^{a} | 15.5^{a} | 7.0 | 25.2 | 14.8^{a} | 41.3^{a} | 18.7 |
| | Fetal skin | 54 | 52 | 96.3 | 44 | 38.6 | 15.9^{ab} | 15.9^{a} | 6.8 | 22.7 | 22.7^{ab} | 40.9^{ab} | 13.6 |
| | fibroblast | | | | | | | | | | | | |
| LiTM | Adult skin | 248 | 229 | 92.3 | 161 | 41.0 | 23.6^{b} | 5.6^{b} | 11.8 | 24.8 | 36.0^{b} | 28.0^{b} | 11.2 |
| | fibroblast | | | | | | | | | | | | |
| Total: Σ | 497 | 471 | 94.8 | 360 | 37.5 | 17.2 | 11.1 | 9.2 | 24.7 | 25.3 | 35.3 | 14.7 | |

*The numbers of injected oocytes shown here are less than those given in the previous column relating to number of injected oocytes because some were used for other experiments; for example, in PCC spindle formation checks.
^{a,b}Letters indicate that figures within the column show a significant difference, i.e. a is significantly different from b (P < 0.05), but ab is not significantly different from a or b (P < 0.05).
†LoTM = Long-tailed Macaque; Macaca fascicularis
LiTM = Lion-tailed Macaque; Macaca silenus
‡RN = reconstructed nucleus

chromosome (PCC); microtubule assembly occurred in 68.5% (37/54) of the reconstructed embryos, and 14.8% (8/54) of them formed the first mitotic spindle normally with 2 poles. Control somatic cells injected into non-enucleated oocytes also formed normal spindles.

TABLE 2
Spindle formation in SCNT embryos at 2 h after cell injection*

| | DNA | | | Percent- |
|---|---|---|---|---|
| Spindle | Condensed | | No. of | age |
| Formation | to PCC | Microtubule Assembly | Oocytes | % |
| Normal | Condensa- | Normal assembly with 2 | 8 | 14.8 |
| spindle | tion | poles | | |
| Abnormal | Condensa- | Normal assembly but im- | 5 | 9.3 |
| Spindle | tion | proper chromosome capture | | |
| | Condensa- | Abnormal assembly | 17 | 31.5 |
| | tion | | | |
| | Condensa- | No assembly | 8 | 14.8 |
| | tion | | | |
| | No Change | Abnormal assembly | 7 | 13.01 |
| No | No Change | No assembly | 9 | 16.7 |
| Spindle | | | | |

*Total oocytes: n = 54.

Pregnancy Outcomes After Embryos Transfer

On day 3, 93 reconstructed embryos (4-10 cell stages) were transferred to 31 LoTM's (same macaques from which the oocytes were collected). The results of SCNT embryos transfer are shown in Table 3. The pregnancy rate was not markedly affected by donor cell type. The pregnancies were confirmed by ultrasound.

TABLE 3
The results of SCNT embryos transfer*

| | | No. of | No. of | |
|---|---|---|---|---|
| | No. of SCNT | Reci- | Preg- | Pregnancy |
| Donor cell type | emb. transferred | pients | nancies | Rate % |
| LiTM Adult Fibroblast | 57 | 18 | 4 | 22.2 |
| LoTM Cumulus | 25 | 9 | 2 | 22.2 |
| LiTM Adult Fibroblast | 3 | 2 | 0 | 0 |
| LoTM Cumulus | 2 | | | |
| LoTM Fetal Fibroblast | 4 | 2 | 1 | 50.0 |
| LoTM Cumulus | 2 | | | |
| TOTAL: Σ | 93 | 31 | 7 | 22.6 |

*Pregnancy was confirmed by ultrasound.

TABLE 4
Details of pregnant M. fasicularis.

| | NT Embryos Transfer | | | Ultrasound | |
|---|---|---|---|---|---|
| Surrogate | | No. of | | Results | |
| ID | Date | Emb. | Donor Cell | Day | GS/FH |
| 168D | Nov. 16, 2001 | 4 | LiTM Fibrob. | 60 | +/+ |
| 9FC0 | Dec. 21, 2001 | 4 | LiTM Fibrob. | 19 | +/+ |
| 9F41 | Jan. 18, 2002 | 1 | LoTM Cumulus | 18 | +/± |
| 6245 | Mar. 08, 2002 | 3 | LoTM Cumulus | 15 | +/± |
| E22E | Jul. 12, 2002 | 2 | LiTM Fibrob | 18 | +/± |
| OFC7 | Aug. 30, 2002 | 4 | LiTM Fibrob. | 15 | +/± |
| 99B7 | Nov. 08, 2002 | 2 | LoTM Fetal | 15 | +/+ |
| | | | Fibrob. | | |
| | | 1 | LoTM Cumulus | | |

The First Cell Cycle of SCNT Reconstructed Embryos

Based on the above data, it appears that the G1/G0 donor nucleus (diploid, 2n) needs to undergo chromosomal changes as with any other cell undergoing mitotic division. Hence the DNA of the somatic cell undergoes condensation (PCC, Pre-matured Condensed Chromosome, 2n) within 2 hours of being introduced into the oocyte, with the formation of a normal spindle in about 14.8% of cases. The majority undergo various abnormal changes, including formation of normal microtubules but improper capture of condensed DNA, formation of an abnormal spindle, absence of microtubule assembly, absence of DNA condensation with an abnormal spindle, and even absence of change. Following activation, with a decline in MPF amongst other signals, mitosis resumes, and the DNA separates normally, or abnormally. After this, the PCC (2n) decondenses to chromatin, the nuclear membrane reforms, the cell goes into G1 and S phase, and the DNA starts to duplicate from 2n to 4n. Culture in cytochalasin B for a few hours after activation at this stage prevents cleavage which results in haploid (1 n) cells, thus resulting in 2 nuclei about 14-16 hours after activation, seen in 11.1% of the reconstructed embryos. Multiple and single nuclei are also seen in 9.2% and 17.2% of cases respectively; the former probably arise from an abnormal spindle, whilst the latter probably arise from absence of DNA separation or absence of microtubule formation. In the next cell cycle, in which a normal spindle reforms, the DNA (4n) re-separates into normal diploid states (2n) or abnormal aneuploid states, sometimes resulting in severe fragmentation.

In this study, 70.4% of somatic cell DNA underwent condensation following introduction into enucleated MII oocytes; microtubules assembled in 68.5% of reconstructed embryo, and 14.8% of these were normal spindles with 2 poles. Without wishing to be bound by theory, it is postulated that consequent spindle formation is influenced by both the donor cell and the oocyte. Poor quality oocytes may not be able to initiate nuclear membrane breakdown of the somatic cell, DNA condensation or microtubule assembly.

Nuclear formation rate in this study was 37.5%, though DNA condensation and microtubule assembly occur in 70% of reconstructed embryos.

The first mitotic division of the reconstructed embryos resulting in equal normal-looking blastomeres was seen in 24.7% of our series. This is higher than formation of normal spindles (14.8%) as well as formation of 2 normal nuclei (11.1%).

The findings disclosed herein demonstrate that somatic cell DNA can condense to form a normal spindle within 2 hours of injection into enucleated oocytes, and that these reconstructed SCNT embryos can result in implantation and pregnancy after transfer into recipient hosts. Nuclear formation, normal division of SCNT embryos and pregnancy after SCNT embryo transfer were not markedly affected by the donor cell, as they were similar among the three different types of donor cells used: cumulus, fetal fibroblast and adult fibroblast.

To date, there has been no successful live-birth with SCNT in non-human primates. Without wishing to be bound by theory, we postulate that this may due to technical problems such as: excessive aspiration of cytoplasm (less than 2% of cytoplasm is removed in the methods of the present invention) and therefore excessive removal of factors essential for proper reprogramming of somatic cell nuclear material; cell injection technique; and activation methods. Culture environment may also be a contributory factor as an optimal medium for SCNT has not been reported. In fact, our data supports the conventional belief that incomplete nuclear re-programming is probably the reason for the lack of SCNT-derived pregnancies in primates.

With the methods of the present invention, we have obtained pregnancies after transfer of SCNT embryos into recipients. This is the first report of pregnancies in non-human primate SCNT.

### INDUSTRIAL APPLICABILITY

The methods of the present invention can be readily used in, for example:
(1) Any method involving the use of enucleated cells, and particularly methods involving the use of oocytes as recipient cells for nuclear transfer;
(2) Any method involving activation of reconstituted vertebrate cells, regardless of how created.
(3) Production of primate (human and non-human) Embryonic Stem Cells (ESC's) for therapy or for creation of models for the study of diseases.

It will be appreciated that, although specific embodiments of the invention have been described herein for the purpose of illustration, various modifications may be made without deviating from the spirit and scope of the invention as defined in the following claims.

1. A method for cell enucleation, comprising removal of the spindle body with a minimal amount of cytoplasm from a cell in metaphase, said method comprising use of polarized light microscopy for visualization of the spindle body during cell enucleation.

2. The method of claim 1, wherein less than about 2% of the cytoplasm is removed from said cell.

3. The method of claim 2, wherein less than about 1% of the cytoplasm is removed.

4. The method of claim 1, wherein the cell is an oocyte.

5. The method of claim 1, wherein the cell, is a vertebrate MII oocyte.

6. The method of claim 1, wherein the cell is a primate MII oocyte.

7. The method of claim 1, wherein said cell is an MII non-human primate oocyte.

8. The method of claim 1, wherein the cell is an oocyte, and the spindle body is removed through a small hole formed in the zona pellucida.

9. The method of claim 8, wherein said small hole is from about 5 μm to about 10 μm.

10. The method of claim 8, wherein said small hole is made by zona drilling using acid Tyrode's solution.

11. The method of claim 10, wherein the pH of the acid Tyrode's solution is about 1.8.

12. The method of claim 1, wherein a needle having an internal diameter of about 6 μm to about 10 μm is used to aspirate the spindle body from the cell.

13. The method of claim 1, wherein a non-spiked needle is used to aspirate the spindle body from the cell.

14. A method for production of an activated reconstructed vertebrate cell, said method comprising:
a. culturing a reconstructed vertebrate cell for a period of time after introduction of the donor nucleus to the recipient cell which is sufficient for the formation of the prematured condensed chromosome and spindle;
b. applying at least one electrical pulse to the reconstructed cell;
c. culturing the reconstructed cell for a period of time sufficient to allow the cell membrane to recover from the electrical pulse; and
d. treating the reconstructed embryo with at least one chemical activator.

15. The method of claim 14, wherein the reconstructed cell is produced by introducing a donor vertebrate nucleus into an enucleated cell.

16. The method of claim 14, wherein step (a) comprises culturing the reconstructed cell for about 1.5 to about 4 hours after introduction of the donor nucleus to the recipient cell.

17. The method of claim 14, wherein step (c) comprises culturing the reconstructed cell for about 1 to about 3 hours after application of the at least one electrical pulse.

18. The method of claim 14, wherein two pulses of direct current at from about 130V/mm to about 180V/mm are applied to the reconstructed cell for from about 40 to about 60 μs for each pulse.

19. The method of claim 14, wherein the reconstructed cell is treated with ethanol as a chemical activator.

20. The method of claim 19, wherein the treatment with ethanol comprises treating the cell with culture medium comprising about 7% v/v ethanol for about 4 to about 7 minutes.

21. The method of claim 14, wherein the reconstructed cell is treated with ionomycin as a chemical activator

22. The method of claim 21, wherein the treatment with ionomycin comprises treating the cell with culture medium comprising about 5 μM ionomycin for about 4 to about 7 minutes.

23. The method of claim 14, further comprising the step of culturing the reconstructed embryo in the presence of at least one ploidy stabilizer.

24. The method of claim 23, wherein the ploidy stabiliser comprises cytochalasin B.

25. The method of claim 24, wherein the reconstructed embryo is cultured in the presence of about 5 μg/ml cytochalasin B.

26. The method of claim 24, wherein the cell is cultured in the presence of cytochalasin B for about 5 to 6 hours.

27. The method of claim 23, wherein the ploidy stabilizer comprises cycloheximide.

28. The method of claim 27, wherein the cell is cultured in the presence of about 10 μg/ml cycloheximide.

29. The method of claim 28 wherein the cell is cultured in the presence of cycloheximide for about 5 to 6 hours.

30. The method of claim 23, wherein the ell is cultured in the presence of about 5 μg/ml cytochalasin B and about 10 μg/ml cycloheximide for about 5 to 6 hours.

31. The method of claim 15, wherein the enucleated cell is prepared by the method of claim 1.

32. The method of claim 15, wherein said enucleated cell is an enucleated oocyte.

33. The method of claim 15, wherein the donor nucleus is a somatic cell nucleus.

34. The method of claim 15, wherein the donor nucleus is introduced into the enucleated cell by direct injection.

35. The method of claim 33, wherein the donor nucleus is introduced into the enucleated cell by direct injection of the donor cell into the cell.

36. The method of claim 15, wherein the donor nucleus is introduced into the enucleated cell by electrofusion of the enucleated cell with the donor cell.

37. The method of claim 15, wherein the donor nucleus is a cumulus or fibroblast cell nucleus.

38. The method of claim 15, wherein the donor nucleus is a quiescent cell nucleus in the G0 or G1 phase.

39. The method of claim 15, wherein said vertebrate is a primate.

40. The method of claim 15, wherein said vertebrate is a non-human primate.

41. A totipotent or pluripotent vertebrate cell isolated from an activated reconstituted vertebrate cell obtained by the method of claim 14.

42. The cell of claim 41, wherein said vertebrate is a primate.

43. The cell of claim 41, wherein said vertebrate is a non-human primate.

44. A method for generating a cloned vertebrate, said method comprising implanting an activated reconstructed vertebrate cell produced by the method of claim 14 into a compatible host uterus.

45. The method of claim 44, wherein said vertebrate is a primate.

46. The method of claim 44, wherein said vertebrate is a non-human primate.

47. A cloned vertebrate generated by the method of claim 44.

48. The cloned vertebrate of claim 47, which is a primate.

49. The cloned vertebrate of claim 47, which is a non-human primate.
